(19) United States
(12) Patent Application Publication
(10) Pub. No.: US 2025/0041389 A1
(43) Pub. Date: Feb. 6, 2025

(54) ENHANCING ABSORPTION OF DIETARY COMPOUNDS TO PROMOTE HEALTH

(71) Applicant: ARIZONA BOARD OF REGENTS ON BEHALF OF THE UNIVERSITY OF ARIZONA, Tucson, AZ (US)

(72) Inventor: Janet Funk, Tucson, AZ (US)

(21) Appl. No.: 18/789,450

(22) Filed: Jul. 30, 2024

Related U.S. Application Data
(60) Provisional application No. 63/517,235, filed on Aug. 2, 2023.

(51) Int. Cl. A61K 38/47; A61K 31/12; C12N 9/24

(52) U.S. Cl. CPC A61K 38/47; A61K 31/12; C12N 9/2402; C12Y 302/01031

(57) ABSTRACT

Methods for enhancing the therapeutic effects of phenols comprising the oral coadministration or sequential oral administration of a phenol and glucuronidase, and compositions for enhancing the therapeutic effects of phenols.

### CROSS-REFERENCE TO RELATED APPLICATIONS

This application is a non-provisional and claims benefit of U.S. Provisional Application No. 63/517,235 filed Aug. 2, 2023, the specification of which is incorporated herein in their entirety by reference.

### STATEMENT REGARDING FEDERALLY SPONSORED RESEARCH OR DEVELOPMENT

This invention was made with government support under Grant No. AR078424 awarded by National Institutes of Health. The government has certain rights in the invention.

### FIELD OF THE INVENTION

The present invention relates to dietary phenols (including phenols formed in the gastrointestinal tract by breakdown of more complex plant derived compounds, such as breakdown of rutin to form quercetin), dietary supplements comprising phenols, and compositions and methods of increasing the bioavailability and/or absorption or reabsorption of the same in the gastrointestinal tract of a subject following oral ingestion of the same.

### BACKGROUND OF THE INVENTION

Many health-promoting plant-derived compounds found in the typical human diet or consumed as dietary supplements are phenolic compounds. Upon consumption, these compounds are rapidly inactivated by the liver via conjugation, where a glucuronide molecule is attached, rendering them inactive. Glucuronidated compounds are, in fact, the primary circulating metabolites of many beneficial phenols, whether from the diet or supplements. Notably, dietary phenols are among the most commonly used supplements in the United States, with usage particularly high among specific populations. For instance, a recent epidemiological study found that women diagnosed with breast cancer frequently use dietary supplements that are metabolized into glucuronidated compounds upon ingestion.

Glucuronidation is a common in vivo metabolic fate of dietary plant-derived phenols, many of which (e.g., quercetin) have beneficial associations with musculoskeletal health, including health span promotion and reduced frailty and bone fracture risk, possibly attributable in part to their elimination of senescent cells promoting age-related declines in bone and muscle (“senolysis”). Examples of compounds that form inactive glucuronides when ingested include turmeric (curcumin), quercetin, fisetin, resveratrol, melatonin, cannabis (tetrahydrocanabinol [THC]), ginger (gingerols), and tart cherry (multiple phenols).

Beta-glucuronidase (GUSB) reverses glucuronidation, reforming the active compound (e.g., curcumin, quercetin, and resveratrol). Blockade of GUSB prevents deconjugation of their bone-inactive glucuronides by GUSB-containing bone marrow cells; stated inversely, the bone-active “aglycone” forms of these compounds can reform in the bone due to local GUSB activity.

Inactive circulating glucuronidated compounds are cleared from the body by secretion back into the gastrointestinal tract via bile (i.e., via enterohepatic circulation). Glucuronidated compounds in the gastrointestinal tract are not readily reabsorbed unless they are deconjugated back into the original form in which they were ingested. Some gut bacteria express enzymes capable of deconjugating glucuronidated compounds from glucuronides. This may allow certain glucuronidated phenols to circulate for longer and at higher levels in the body.

Additionally, recent research suggests that at least one endogenously-produced enzyme in the human body, found at very high levels in bone, is capable of reversing glucuronide conjugation, thus removing the glucuronide and reforming an active compound, including active phenols. This process may explain how certain dietary phenols are able to prevent bone loss (for example, that occurs with aging), along with providing other health benefits, even though the circulating forms of these compounds are inactive due to being glucuronidated.

### BRIEF SUMMARY OF THE INVENTION

It is an objective of the present invention to provide compositions and methods that allow for increased bioavailability, absorption, and/effectively increases in the gastrointestinal tract following oral ingestion of phenols, as specified subject's gastrointestinal tracts of the invention are given in the dependent claims. Embodiments of the present invention can be freely combined with each other if they are not mutually exclusive.

In some aspects, the present invention features a method of increasing either one or both of the bioavailability or reabsorption of a phenol in the gastrointestinal tract of a subject in need thereof. The method may comprise orally co-administering or sequentially orally administering a therapeutic amount of glucuronidase and a phenol to the subject.

In other aspects, the present invention features a composition comprising glucuronidase and a phenol. Said composition may effectively increase at least one of the bioavailability, absorption, or reabsorption of the phenol in a subject's gastrointestinal tract upon oral administration of the composition to the subject.

One of the unique and inventive technical features of the present invention is the co-administration of glucuronidase and a phenol. Without wishing to limit the invention to any theory or mechanism, it is believed that the technical feature of the present invention advantageously provides for increased bioavailability, absorption, and/or reabsorption of phenols within the gastrointestinal tract.

Again, without wishing to limit the invention to any theory or mechanism, it is also believed that another advantageous technical feature of the invention is that the co-administration of glucuronidase and phenols does not significantly interfere with the body's removal of toxic compounds. This is a potential concern with administering glucuronidases via the gastrointestinal tract, as the clearance of glucuronides through enterohepatic circulation is a key route for the elimination of toxins. However, the present invention increases glucuronidase levels in a temporary manner that coincides with the oral intake of beneficial compounds. This minimizes or eliminates the potential negative effects on the body's ability to remove toxins through enterohepatic circulation.

As an analogy, a lactose-intolerant individual only needs to take oral lactase supplementation when consuming dairy products. Similarly, an individual consuming health-promoting phenols, either through their diet or as dietary supplements, would only need to consume glucuronidase when their diet is high in phenols or when taking phenol-rich supplements.

None of the presently known prior references or work has the unique, inventive technical feature of the present invention. In fact, prior references teach away from the present invention. For instance, earlier research concluded that co-administering an enzyme would be impractical due to the significant challenges associated with enzyme delivery. Oral delivery of enzymes faces major technical obstacles, primarily because these enzymes are susceptible to proteolytic degradation in the gastrointestinal (GI) tract, especially in the stomach. However, the Inventors have discovered that the present invention surprisingly allows for the oral administration of enzymes, including beta-glucuronidase, which retain their enzymatic activity after passing through the proteolytic sections of the GI tract, such as the stomach and duodenum.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows oral co-administration of an enzyme (GUSB) with curcumin, a phenol (more specifically, a polyphenol) and the active ingredient in turmeric dietary supplements, which is the top-selling botanical supplement in the US, increases the amount of active curcumin in the circulation, and in bone, a key target organ for beneficial effects of curcumin. Note-enzyme levels in the body are unchanged with this administration. Effects all occur in the gut, increasing curcumin absorption. Effects on the bioavailability of other dietary phenols with health benefits would be expected to be similar.

FIG. 2 shows the normal elimination of glucuronidated phenols.

FIG. 3 shows that increasing GUSB levels in the gut at the time of phenol ingestion increases reabsorption and, thus, the bioavailability of phenols per dose.

### DETAILED DESCRIPTION OF THE INVENTION

Before the present compounds, compositions, and/or methods are disclosed and described, it is to be understood that this invention is not limited to specific synthetic methods or to specific compositions, as such may, of course, vary. It is also to be understood that the terminology used herein is for the purpose of describing particular embodiments only and is not intended to be limiting.

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. The term “comprising” means that other elements can also be present in addition to the defined elements presented. The use of “comprising” indicates inclusion rather than limitation. Stated another way, the term “comprising” means “including principally, but not necessary solely”. Furthermore, variation of the word “comprising”, such as “comprise” and “comprises”, have correspondingly the same meanings. In one respect, the technology described herein related to the herein described compositions, methods, and respective component(s) thereof, as essential to the invention, yet open to the inclusion of unspecified elements, essential or not (“comprising”).

Although methods and materials similar or equivalent to those described herein can be used to practice or test the disclosed technology, suitable methods and materials are described below. The materials, methods, and examples are illustrative only and not intended to be limiting.

The term “phenol” means chemical compounds containing one or more hydroxyl groups bonded directly to an aromatic hydrocarbon group, and includes, but is not limited to, polyphenols (e.g., tannins, flavonoids, etc.), simple phenols (e.g., phenolic acids, etc.), miscellaneous phenolic compounds. Tannins include, but are not limited to, hydrolysable tannins (e.g., polymers of phenolic acid derivatives) and condensed tannins (e.g., polymers of catechins and epicatechins). Flavonoids include, but are not limited to, flavones, flavonols, aurones, chalcones, flavanones, isoflavanones, flavanols, anthocyanins, etc. Phenolic acids include, but are not limited to, dydroxy cinnamic acid derivates, hydroxy benzoic acid derivatives, etc. Miscellaneous phenolic compounds include, but are not limited to, lignans, coumarins, lignins, resveratrol, etc.

A “subject” is an individual and includes, but is not limited to, a mammal (e.g., a human, horse, pig, rabbit, dog, sheep, goat, non-human primate, cow, cat, guinea pig, or rodent), a fish, a bird, a reptile or an amphibian. The term does not denote a particular age or sex. Thus, adult and newborn subjects, as well as fetuses, whether male or female, are intended to be included. A “patient” is a subject afflicted with a disease or disorder.

The terms “treating” or “treatment” refers to any indicia of success or amelioration of the progression, severity, and/or duration of a disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; or improving a patient's physical or mental well-being.

The terms “manage,” “managing,” and “management” refer to preventing or slowing the progression, spread, or worsening of a disease or disorder, or of one or more symptoms thereof. In certain cases, the beneficial effects that a subject derives from a prophylactic or therapeutic agent do not result in a cure of the disease or disorder.

The term “effective amount” as used herein refers to the amount of a therapy (e.g., a postbiotic) which is sufficient to reduce and/or ameliorate the severity and/or duration of a given disease, disorder, or condition and/or a symptom related thereto. This term also encompasses an amount necessary for the reduction or amelioration of the advancement or progression of a given disease, disorder or condition, reduction or amelioration of the recurrence, development or onset of a given disease, disorder or condition, and/or to improve or enhance the prophylactic or therapeutic effect(s) of another therapy. In some embodiments, “effective amount” as used herein also refers to the amount of therapy provided herein to achieve a specified result.

As used herein, and unless otherwise specified, the term “therapeutically effective amount” of therapy described herein is an amount sufficient to provide a therapeutic benefit in the treatment or management of a given disease, or to delay or minimize one or more symptoms associated with said disease. A therapeutically effective amount of a composition described herein means an amount of therapeutic agent, alone or in combination with other therapies, which provides a therapeutic benefit in the treatment or management of a disease. The term “therapeutically effective amount” can encompass an amount that improves overall therapy, reduces or avoids symptoms or causes, or enhances the therapeutic efficacy of another therapeutic agent.

The terms “administering” and “administration” refer to methods of providing pharmaceutical preparation to a subject. Such methods are well known to those skilled in the art and include, but are not limited to, administering the compositions orally, intranasally, by intramuscular injection, by intraperitoneal injection, intrathecally, transdermally, extracorporeally, topically or the like.

As used herein, the terms “co-administration” and “co-administering” refer to the administration of at least two agent(s) (e.g., glucuronidase and phenols) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

Pharmaceutical peptide compositions for oral administration include, but are not limited to, powders or granules, suspensions or solutions in water or non-aqueous media, pills, lozenges, capsules, sachets, or tablets. Thickeners, flavorings, diluents, emulsifiers, dispersing aids, or binders may be desirable. A person of skill, monitoring a subject's clinical response, can adjust the frequency of administration and dosage of the medication according to methods known in the art.

As described above, the compositions can be administered to a subject in a pharmaceutically acceptable carrier. By “pharmaceutically acceptable,” it is meant a material that is not biologically or otherwise undesirable, i.e., the material may be administered to a subject without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

Pharmaceutical carriers are known to those skilled in the art. These most typically would be standard carriers for administration of drugs to humans, including solutions such as sterile water, saline, and buffered solutions at physiological pH. Typically, an appropriate amount of a pharmaceutically acceptable salt is used in the formulation to render the formulation isotonic. Examples of the pharmaceutically acceptable carrier include, but are not limited to, saline, Ringer's solution and dextrose solution. The pH of the solution is preferably from about 5 to about 8, and more preferably from about 7 to about 7.5. Further carriers include sustained release preparations such as semi-permeable matrices of solid hydrophobic polymers containing the disclosed compounds, which matrices are in the form of shaped articles, e.g., films, liposomes, microparticles, or microcapsules. It will be apparent to those persons skilled in the art that certain carriers can be more preferable depending upon, for instance, the route of administration and concentration of composition being administered. Other compounds can be administered according to standard procedures used by those skilled in the art.

Pharmaceutical formulations can include additional carriers, as well as thickeners, diluents, buffers, preservatives, surface active agents and the like in addition to the compounds disclosed herein.

In some embodiments, the glucuronidase and phenol are administered orally. In some embodiments, the glucuronidase and phenol are administered to the patient on a twice daily basis, once daily basis, on an alternating daily basis, on a weekly basis, on a monthly basis, or at any interval in between. A person of skill, monitoring a subject's clinical response and improvement, can determine the frequency of administration of the medication by methods known in the art. In some embodiments, the glucuronidase and phenol are administered every third day. In some embodiments, the glucuronidase and phenol are administered every other day. In some embodiments, the glucuronidase and phenol are administered daily.

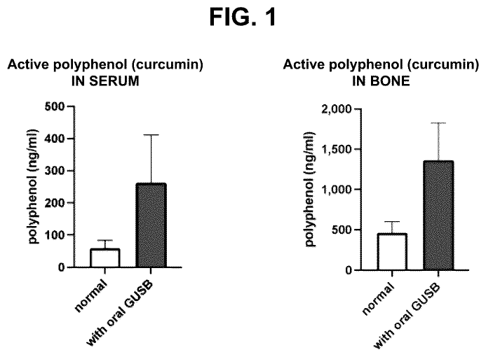

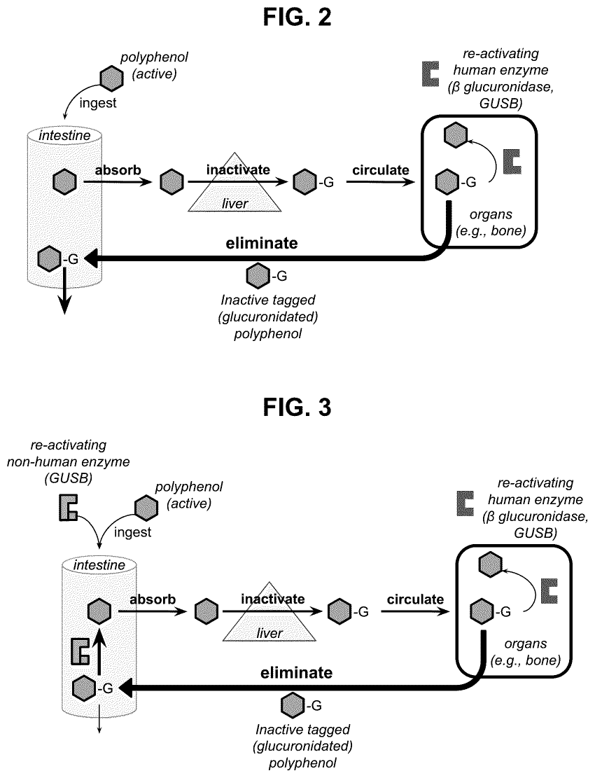

Referring now to FIGS. 1-3, the present invention features methods and compositions for increasing at least one of bioavailability, absorption, and reabsorption of the phenol in a gastrointestinal tract of a subject.

The present invention may feature a composition comprising glucuronidase and a phenol. In some embodiments, the composition is effective at increasing either one or both bioavailability or reabsorption of a phenol in the gastrointestinal tract of a subject upon oral administration of the composition to the subject.

In other embodiments, the present invention features a first composition comprising a glucuronidase and a second composition comprising a phenol. In some embodiments, when administered sequentially or simultaneously, the compositions (e.g., the first composition and second composition) are effective at increasing either one or both bioavailability or reabsorption of the phenol (e.g., the composition comprising a phenol; e.g., the second composition) in the gastrointestinal tract of a subject upon oral administration to the subject.

In certain embodiments, the present invention features a composition comprising glucuronidase and a glycoside (or ester) of a phenol. In other embodiments, the present invention features a first composition comprising a glucuronidase and a second composition comprising a glycoside (or ester) of a phenol.

The present invention may also feature a method of increasing either one or both bioavailability or reabsorption of a phenol in the gastrointestinal tract of a subject in need thereof. In some embodiments, the method comprises simultaneously co-administering a therapeutic amount of glucuronidase and a phenol orally to the subject. In some embodiments, the method comprises simultaneously co-administering a therapeutic amount of a first composition comprising a glucuronidase and a second composition comprising a phenol orally to the subject. In further embodiments, the method comprises orally co-administering a therapeutic amount of a composition comprising a glucuronidase and a phenol to the subject.

In some embodiments, the method comprises sequentially co-administering a therapeutic amount of glucuronidase and a phenol orally to the subject. In other embodiments, the method comprises sequentially co-administering a therapeutic amount of a first composition comprising a glucuronidase and a second composition comprising a phenol orally to the subject.

In some embodiments, the method comprises administering a therapeutic amount of the glucuronidase first, followed by a therapeutic amount of the phenol to the subject. In other embodiments, the method comprises first administering a therapeutic amount of a composition comprising a glucuronidase, followed by a composition comprising a phenol to the subject.

In some embodiments, the method comprises administering a therapeutic amount of the phenol first, followed by a therapeutic amount of the glucuronidase to the subject. In other embodiments, the method comprises first administering a therapeutic amount of a composition comprising a phenol, followed by a composition comprising a glucuronidase to the subject.

Without wishing to limit the present invention to any theory or mechanism, it is believed that the compositions described herein (e.g., glucuronidase and a phenol) must be administered orally to work and cause the reuptake of phenol from the gut.

In some embodiments, the glucuronidase is beta-glucuronidase. In other embodiments, the glucuronidase is alpha-glucuronidase.

In some embodiments, the glucuronidase used herein is derived in a species-agnostic manner, such as from yeast, fungi, snails (e.g., Helix pomatia), or other appropriate molluscs. In some embodiments, the glucuronidase used herein is produced by or isolated from bacteria. In other embodiments, the glucuronidase used herein is produced by or isolated from yeast or fungi. Recombinant genetic techniques may also be used to produce glucuronidase. For example, recombinant GUSB can also be generated by cloning a specific GUSB gene (such as GusA), including variants found in humans, into a host cell such as bacteria or yeast. Variants of GUSB may be selected based on optimized activity under specific pH or temperature conditions and for stability during various modes of delivery.

Additionally, in certain embodiments, the glucuronidase is derived from bacteria or through recombinant genetic technology. In some embodiments, naturally occurring glucuronidase (GUSB) produced by bacteria or yeast may be isolated. In other embodiments, the glucuronidase is a recombinant human GUSB.

In some embodiments, the GUSB may be modified. Modifications may include, but are not limited to, modifying various isoenzymes of glucuronidase from wild-type sequences to mutant or modified sequences. For example, modifications may enhance proteolytic stability or increase activity within specific pH ranges. In some embodiments, the aforementioned modifications may be achieved by engineering the gene or by identifying naturally occurring variants in bacteria, yeast, or snails. Alternatively, the properties of naturally occurring variants may be used to design and modify the GUSB gene to create an enzyme with similar properties.

In some embodiments, the glucuronidase has sufficient stability to remain active in a small intestine after passing through the stomach of the subject. In some embodiments, the glucuronidase has increased activity at neutral or near-neutral pH levels, as compared to wild-type human glucuronidase. In some embodiments, the glucuronidase has increased activity at about pH 6, as compared to wild-type human glucuronidase. In some embodiments, the glucuronidase has increased stability, as compared to wild-type human glucuronidase.

Without wishing to limit the present invention to any theory or mechanism, it is believed that glucuronidase facilitates the conversion of a phenol metabolite into its therapeutically active form. For example, glucuronidase catalyzes the transformation of a phenol metabolite (e.g., glucuronidated phenol) back into the active phenol.

In some embodiments, the phenol is a tannin, phenolic acid, stilbene, lignan, flavonoid. In some embodiments, the tannin is tannic acid. In some embodiments, the phenolic acid is caffeic acid, ferulic acid, benzoic acid, cinnamic acid, or gallic acid. In some embodiments, the stilbene is resveratrol. In some embodiments, the flavonoid is a flavone, a flavonol, a flavanol, a flavanone, or an isoflavone. In some embodiments, the flavonol is quercetin or fisetin. In some embodiments, the flavanol is catechin. In some embodiments, the flavanone is hesperetin. In some embodiments, the isoflavone is daidzein. In some embodiments, the flavonoid is a proanthocyanidin, an anthocyanidin, or an anthocyanin. In some embodiments, the anthocyanin is cyanidin. In some embodiments, the phenol is curcumin, a gingerol or a shogaol. The present invention is not limited to the aforementioned phenols and may also include any phenol that undergoes glucuronidation upon ingestion, provides health benefits, or both.

In certain embodiments, the phenol is a tannin, phenolic acid, stilbene, lignan, and flavonoid that is glucuronidated upon ingestion.

In some embodiments, the glucuronidase is provided in an enteric coating. In some embodiments, the phenol is provided in an enteric coating. In other embodiments, the glucuronidase and the phenol are provided in an enteric coating. In certain embodiments, the composition comprising a glucuronidase and phenol is provided in an enteric coating. In some embodiments, the first composition comprising a glucuronidase may be provided in an enteric coating, while separately or additionally, the second composition comprising a phenol may also be provided in an enteric coating.

In some embodiments, the glucuronidase is encapsulated in a micelle. In some embodiments, the phenol is encapsulated in a micelle. In other embodiments, the glucuronidase and the phenol are encapsulated in a micelle. In certain embodiments, the composition comprising a glucuronidase and a phenol is encapsulated in a micelle. In some embodiments, the first composition comprising a glucuronidase may be encapsulated in a micelle, while separately or additionally, the second composition comprising a phenol may also be encapsulated in a micelle.

In some embodiments, the glucuronidase is encapsulated in a nanoparticle. In some embodiments, the phenol is encapsulated in a nanoparticle. In other embodiments, the glucuronidase and the phenol are encapsulated in a nanoparticle. In certain embodiments, the composition comprising a glucuronidase and a phenol is encapsulated in a nanoparticle. In some embodiments, the first composition comprising a glucuronidase may be encapsulated in a nanoparticle, while separately or additionally, the second composition comprising a phenol may also be encapsulated in a nanoparticle.

The compositions (e.g., glucuronidase and a phenol) described herein are not limited to the aforementioned delivery methods and may utilize any appropriate delivery method well known in the art.

In some embodiments, the methods and compositions described herein are effective in reducing the enterohepatic circulation of the phenol administered to the subject. In other embodiments, the methods and compositions described herein are effective to increase the half-life of the phenol administered to the subject. In some embodiments, the methods described herein are effective at increasing the bioavailability of the phenol administered to the subject.

In some embodiments, the methods and compositions described herein are effective for modulating either one or both of the pharmacokinetics or the metabolism of the phenol.

In some embodiments, the methods and compositions described herein are effective in reducing insulin resistance, inflammation, and/or frailty. In some embodiments, the methods and composition described herein are effective at maintaining joint health. In other embodiments, the methods and composition described herein are effective at preventing arthritis, rheumatoid arthritis, osteoarthritis, or a combination thereof.

Methods and compositions described herein may also be effective maintaining metabolic health and reducing risk of diabetes. For example, the methods and compositions described herein may be effective at reducing the risk or preventing diabetes, prediabetes, type 2 diabetes, or both.

The methods and compositions described herein may improve and/or maintain bone health. In some embodiments, the methods and compositions described herein prevent the progression of bone metastases. In other embodiments, the methods and compositions described herein reduce the risk of bone metastasis. Additionally, in other embodiments, the methods and compositions described herein may prevent breast cancer metastases. For example, the Inventors surprisingly found that curcuminoids, such as curcumin, inhibit TGF-β signaling in human breast cancer cells and mitigate osteolysis in breast cancer bone metastasis models.

Additionally, the methods and compositions described herein may prevent post-menopausal bone loss. For example, Inventors have found that curcuminoids (e.g., curcumin) can prevent bone loss in an FDA-approved model for testing osteoporosis treatments (ovariectomized rats) by decreasing bone-resorbing osteoclasts. In some embodiments, other phenols, e.g., quercetin and resveratrol, may be used to prevent post-menopausal bone loss.

The methods and compositions described herein may also mitigate joint inflammation associated with arthritis. For instance, the Inventors have demonstrated that curcuminoids (e.g., curcumin), gingerols, and shogaols can prevent joint inflammation in a rheumatoid arthritis model. In other embodiments, curcuminoids (e.g., curcumin) may alleviate joint pain in osteoarthritis. Additionally, ellagitannins may be used to reduce inflammation in arthritis.

In some embodiments, the methods and compositions described herein may also prevent the loss of muscle and bone associated with aging or other diseases, such as breast cancer or diabetes. For example, curcumin, quercetin, fisetin, shogaol may reduce senescent cells that drive age-related loss of muscle and bone.

Furthermore, the methods and compositions described herein may be effective in reducing glucose levels and improving insulin sensitivity in individuals with obesity and/or type two diabetes. For example, curcumin and cocoa flavanols, such as epicatechin, quercetin, and anthocyanin, may improve insulin sensitivity.

### EXAMPLES

The following are non-limiting examples of the present invention. It is to be understood that said examples are not intended to limit the present invention in any way. Equivalents or substitutes are within the scope of the present invention.

### Example 1

Experimental Methods: Administer 140 U/g body weight of GUSB (E. coli-derived [Sigma G840]) orally via gavage (vs vehicle) to 10-week-old male C57BI6 mice. Five minutes later, administer curcumin (500 mg/kg) orally via gavage. Thirty minutes post curcumin (time of curcumin Cmax), harvest serum and bone for assay of curcumin metabolites (e.g. curcumin and glucuronidated-curcumin using established methods. Note: curcumin is immediately glucuronidated by the liver upon ingestion such that very little bioactive free curcumin is found in the circulation. Curcumin metabolites assayed as per published methods.

### Example 2

A 52-year-old male patient suffers from insulin resistance and is prediabetic. His doctor recommends taking phenol and glucuronidase supplements orally and daily for six months in addition to standard treatments. This patient is examined later by his doctor, including a full lab workup, and his insulin resistance is decreased.

### Example 3

A 61-year-old male patient suffers from chronic joint inflammation. His doctor recommends taking phenol and glucuronidase supplements orally and daily for one year. Upon re-examination, including a full lab workup, his chronic joint inflammation shows a marked improvement.

### Example 4

A 53-year-old female patient is diagnosed with prediabetes. Her doctor recommends taking phenol and glucuronidase supplements sequentially, each orally and daily, for six months. Upon re-examination, including a full lab workup, her prediabetes shows improvement, indicated by decreased fasting blood glucose and A1c levels.

### Example 5

A 38-year-old male patient suffers from type 2 diabetes. His doctor recommends phenol and glucuronidase supplements in addition to standard treatment, advising that the phenol and glucuronidase supplements should be taken together simultaneously, orally and daily for one year. Following a subsequent examination, including a comprehensive lab workup, his type 2 diabetes shows improvement, indicated by decreased fasting blood glucose and A1c levels.

### Example 6

A 66-year-old female patient suffers from rheumatoid arthritis. Her doctor recommends phenol and glucuronidase supplements, to be taken orally and daily for one year. Additionally, the patient has heard from a friend who maintains joint health with these supplements. Encouraged by this, the patient decides to incorporate these supplements into her daily routine. Following a subsequent examination, including a full lab workup, her rheumatoid arthritis shows improvement, indicated by decreased joint inflammation and pain.

### Example 7

A 69-year-old male patient suffers from osteoarthritis. His doctor recommends phenol and glucuronidase supplements to be taken orally and once daily for six months. This patient also knows a friend who successfully improved their osteoarthritis using these supplements, prompting him to incorporate them into his routine alongside standard treatments. Following a subsequent examination, including a full lab workup, his osteoarthritis shows improvement, indicated by decreased joint inflammation and pain.

### Example 8

A 55-year-old female patient suffers from bone loss and is diagnosed pre-osteoporotic. Her doctor recommends taking phenol and glucuronidase supplements orally and daily for six months, alongside calcium and vitamin D supplements. Following a subsequent examination, including a full lab workup, her bone loss shows improvement, indicated by increased bone density on DEXA scan.

### Example 9

A 79-year-old female patient is diagnosed with metastatic lung cancer, with noted bone metastases. Her doctor recommends phenol and glucuronidase supplements to be taken orally and twice daily for six months, alongside standard treatment. Following a subsequent examination, including a full lab workup, she finds that her bone metastases are improving, indicated by decreased quantity and size. Her doctor advises her to continue with phenol and glucuronidase treatment for another six months.

### Example 10

A 66-year-old female patient diagnosed with metastatic breast cancer limited to bone is progressing with standard drug treatments. Her doctor recommends the addition of phenol and glucuronidase supplements to be taken orally and twice daily for six months. This patient is examined later by her doctor, including a full workup, and finds that her breast cancer is no longer progressing, as indicated by bone imaging and liquid biopsy for circulating tumor DNA. Her doctor instructs her to continue with her supplements of phenol and glucuronidase for another six months.

### Example 11

A 42-year-old pre-menopausal female patient is in generally good health but worries about her bone health as she ages and approaches menopause. Her doctor recommends that she starts taking an oral phenol and glucuronidase supplement once daily. Encouraged by the success her friend is having in maintaining bone health with this oral supplement, the patient decides to incorporate the supplement into her routine. She visits her doctor again for her annual checkup, including a full lab workup, and her bone health is found to be good and likely improved, indicated by healthy levels of bone density on DEXA scan.

### Example 12

An 81-year-old female patient is diagnosed with metastatic melanoma, with noted bone metastases. Her doctor recommends adding phenol and glucuronidase supplements to her standard melanoma treatments, to be taken orally and twice daily for three months. These supplements are to be taken sequentially, with phenol and glucuronidase taken one right after the other. Following a subsequent examination, including a full lab workup, the progression of her bone metastases is halted, indicated by no new bone metastases. Her doctor advises her to continue with the phenol and glucuronidase treatment for another six months.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. A composition comprising a glucuronidase and a phenol, wherein the glucuronidase is effective at increasing either one or both bioavailability or reabsorption of the phenol in a gastrointestinal tract of a subject upon oral administration of the composition to the subject.

2. The composition of claim 1, wherein the composition is provided in an enteric coating.

3. The composition of claim 1, wherein the composition comprises the glucuronidase and the phenol encapsulated in a micelle.

4. The composition of claim 1, wherein the glucuronidase is produced by or isolated from bacteria.

5. The composition of claim 1, wherein the glucuronidase is produced by recombinant genetic technology.

6. The composition of claim 1, wherein the glucuronidase is beta-glucuronidase.

7. The composition of claim 1, wherein the glucuronidase has increased stability, as compared to wild-type human glucuronidase.

8. The composition of claim 1, wherein the phenol is a tannin, phenolic acid, stilbene, lignan, or flavonoid.

9. The composition of claim 1, wherein the phenol comprises a glycoside or ester of the phenol.

10. The composition of claim 1, wherein the glucuronidase is involved in the transformation of a metabolite of the phenol back into the phenol.

11. A method of maintaining joint health and/or preventing arthritis, rheumatoid arthritis, and/or osteoarthritis; the method comprising orally co-administering the composition according to claim 1 to the subject.

12. A method of maintaining metabolic health and/or reducing risk of diabetes, prediabetes and/or type 2 diabetes, the method comprising orally co-administering the composition according to claim 1 to the subject.

13. A method of maintaining bone health and/or reducing risk of bone metastases, the method comprising orally co-administering the composition according to claim 1 to the subject.

14. A method of increasing either or both bioavailability and reabsorption of a phenol in a gastrointestinal tract of a subject in need thereof, the method comprising orally co-administering a therapeutic amount of a glucuronidase and a phenol to the subject.

15. The method of claim 14, wherein the glucuronidase and the phenol are co-administered simultaneously.

16. The method of claim 14, wherein the glucuronidase and the phenol are co-administered sequentially.

17. The method of claim 14, wherein either the glucuronidase, the phenol, or both are provided in an enteric coating.

18. The method of claim 14, wherein either the glucuronidase, the phenol, or both are encapsulated in a micelle.

19. The method of claim 14, wherein the phenol is a tannin, phenolic acid, stilbene, lignan, or flavonoid.

20. The method of claim 14, wherein the method prevents prediabetes, type 2 diabetes, rheumatoid arthritis, osteoarthritis, bone loss, bone disease, bone metastases, or breast cancer.
